(19) United States
(12) Patent Application Publication
(10) Pub. No.: US 2025/0040897 A1
(43) Pub. Date: Feb. 6, 2025

(54) Filter Control for a Medical Imaging System

(71) Applicant: Siemens Healthineers AG, Forchheim (DE)

(72) Inventors: Li Qin Huo, Shenzhen (CN); Jian Qiao Zhang, Shenzhen (CN)

(73) Assignee: Siemens Healthineers AG, Forchheim (DE)

(21) Appl. No.: 18/786,747

(22) Filed: Jul. 29, 2024

(30) Foreign Application Priority Data
Aug. 1, 2023 (CN) 202310963797.4

(51) Int. Cl. A61B 6/06; A61B 6/00; A61B 6/58

(52) U.S. Cl. CPC A61B 6/06; A61B 6/547; A61B 6/588

(57) ABSTRACT

An imaging system and a control method and apparatus therefor, a computer medium, and a program product. The medical imaging system includes an examination component operable to align a ray field with a target region of a test subject. The method includes: acquiring contour information of the test subject and initial position information of the test subject relative to the examination component; acquiring real-time position information of the examination component; determining a part of the target region that is located in the ray field based on the contour information of the test subject, the initial position information of the test subject relative to the examination component, and the real-time position information of the examination component; determining a target attitude of a filter in a collimator assembly of the medical imaging system based on said part; and controlling the filter to move toward the target attitude.

### TECHNICAL FIELD

The present disclosure relates to the technical field of medical equipment, in particular to a filter control method and apparatus for a medical imaging system, an imaging method for a medical imaging system, a medical imaging system, a non-transitory computer-readable storage medium, and a computer program product.

### BACKGROUND

Medical imaging systems (e.g., magnetic resonance systems, computerized tomography systems, digital subtraction angiography systems, etc.) can non-invasively acquire and process images of tissues inside the human body. Medical imaging systems may be used for interventional therapy, which involves the introduction of medical equipment into the body of a test subject under the guidance of a medical imaging system so as to perform an operation on a target region in the body of the test subject.

The method described in this section is not necessarily a previously envisaged or used method. Unless otherwise stated, it should not be assumed that any method described in this section is regarded as prior art simply because it is included in this section. Similarly, unless otherwise stated, problems mentioned in this section should not be regarded as having been generally acknowledged in any prior art.

### SUMMARY

According to one aspect of the present disclosure, a filter control method for a medical imaging system is provided, the medical imaging system comprising at least one examination component for aligning a ray field with a target region of a test subject. The method comprises: acquiring contour information of the test subject and initial position information of the test subject relative to the examination component; acquiring real-time position information of the examination component; determining a part of the target region that is located in the ray field on the basis of the contour information of the test subject, the initial position information of the test subject relative to the examination component, and the real-time position information of the examination component; determining a target attitude of a filter in a collimator assembly of the medical imaging system on the basis of said part; and controlling the filter to move toward the target attitude.

According to another aspect of the present disclosure, an imaging method for a medical imaging system is provided, comprising: using the method described above to control the filter; in response to having controlled the filter to move toward the target attitude, acquiring an actual attitude of the filter; and in response to determining that a relationship between the actual attitude and the target attitude meets a preset condition, performing imaging of part of the target region.

According to another aspect of the present disclosure, a filter control apparatus for a medical imaging system is provided, the medical imaging system comprising at least one examination component for aligning a ray field with a target region of a test subject. The apparatus comprises: a first acquisition unit, configured to acquire contour information of the test subject and initial position information of the test subject relative to the examination component; a second acquisition unit, configured to acquire real-time position information of the examination component; a first determining unit, configured to determine a part of the target region that is located in the ray field on the basis of the contour information of the test subject, the initial position information of the test subject relative to the examination component, and the real-time position information of the examination component; a second determining unit, configured to determine a target attitude of a filter in a collimator assembly of the medical imaging system on the basis of said part; and a motion control unit, configured to control the filter to move toward the target attitude.

According to another aspect of the present disclosure, a medical imaging system is provided, comprising: at least one processor; and a memory in communicative connection with the at least one processor, wherein the memory stores a computer program which, when executed by the at least one processor, realizes the method described above.

According to another aspect of the present disclosure, a non-transitory computer-readable storage medium storing a computer program is provided, wherein the computer program, when executed by a processor, realizes the method described above.

According to another aspect of the present disclosure, a computer program product is provided, comprising a computer program which, when executed by a processor, realizes the method described above.

According to aspects of the present disclosure, firstly, there is no need for an operator to manipulate a control lever manually to control the filter in the collimator assembly, so the target attitude of the filter that is finally determined is not restricted by the operator's skill or experience level, and has higher efficiency and accuracy; secondly, there is no need to perform pre-imaging of the test subject, so the radiation dose received by the test subject can be lowered, reducing harm to the test subject's health.

It should be understood that the content described in this section is not intended to identify key or important features of aspects of the present disclosure, and not intended to limit the scope of the present disclosure. Other features of the present disclosure will become easy to understand through the following description.

### BRIEF DESCRIPTION OF THE DRAWINGS

The drawings show aspects demonstratively and form part of this specification, being used together with the textual description of this specification to explain exemplary ways of implementing aspects. The aspects shown merely serve an illustrative purpose and do not limit the scope of the claims. In all of the drawings, identical reference labels denote similar but not necessarily identical key elements.

Aspects of the present disclosure are described in detail below with reference to the accompanying drawings to give those skilled in the art a clearer understanding of the abovementioned and other features and advantages of the present disclosure. In the drawings:

FIG. 1 shows a subtraction angiography image obtained using a relevant technique.

FIG. 2 shows a flow chart of a filter control method for a medical imaging system according to an aspect of the present disclosure.

FIG. 3 shows a flow chart of a partial process of a filter control method for a medical imaging system according to an aspect of the present disclosure.

FIGS. 4a and 4b, respectively, show schematic drawings of virtual system models according to some exemplary aspects of the present disclosure, viewed from different angles.

FIG. 5 shows a flow chart of a partial process of a filter control method for a medical imaging system according to an aspect of the present disclosure.

FIG. 6 shows a flow chart of an imaging method for a medical imaging system according to an aspect of the present disclosure.

FIG. 7 shows a subtraction angiography image obtained using the imaging method according to an aspect of the present disclosure.

FIG. 8 shows a schematic block diagram of a filter control apparatus for a medical imaging system according to an aspect of the present disclosure.

FIG. 9 is a block diagram showing an exemplary electronic device applicable to an exemplary aspect.

### DETAILED DESCRIPTION

Demonstrative aspects of the present disclosure are described below with reference to the drawings, including various details of aspects of the present disclosure to assist understanding, but these should be regarded as merely demonstrative. Thus, those skilled in the art should recognize that various changes and modifications could be made to the aspects described here without deviating from the scope of the present disclosure. Similarly, for clarity and conciseness, descriptions of commonly known functions and structures are omitted in the descriptions below.

In the present disclosure, unless otherwise stated, the use of the terms “first,” “second,” etc., to describe various key elements is not intended to define a positional relationship, time sequence relationship, or importance relationship between these key elements; such terms are merely used to distinguish one element from another. In some examples, a first key element and a second key element may refer to the same instance of the element in question, whereas in some cases, based on the description in the context, they may also denote different instances.

Terms used in the descriptions of the various examples in the present disclosure are merely intended to describe specific examples and are not intended to impose restrictions. Unless clearly indicated otherwise in the context, if the quantity of a key element is not specifically defined, the key element may be one or more. In addition, the term “and/or” used in the present disclosure covers any one of the listed items and all possible combinations.

As stated above, in interventional surgery, a medical imaging system may be used to observe the situation inside a test subject's body in real time. In an example, the medical imaging system may be equipped with a C-arm X-ray machine; by operating a control lever, an operator can control movement of the C-arm X-ray machine about different axes, so as to align an X-ray emission position and orientation with a target region of the test subject. In this way, ray imaging of the target region can be performed. In some scenarios, the emitted rays pass not only through the target region but also through a region surrounding the target region, and consequently, the result of ray imaging not only includes an image of the target region but might also include an image of the region surrounding the target region. When the surrounding region is a cavity or of lower density, the image of the surrounding region might suffer from over-exposure, which will affect normal observation of the target region.

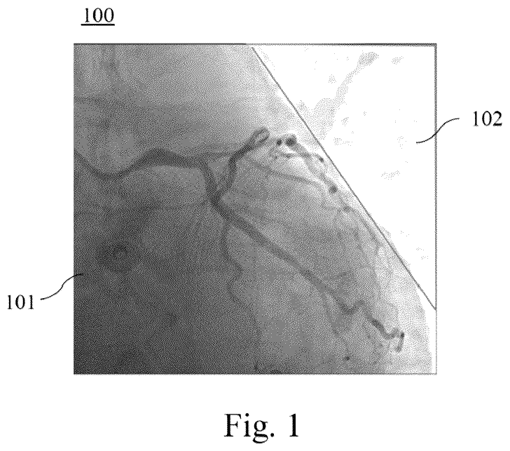

For example, in a scenario in which the medical imaging system is used to perform interventional surgery on a vascular system, the medical imaging system can assist with subtraction angiography. In the process of subtraction angiography, a contrast agent clearly visible in X-ray images may be applied to the blood circulation system of the test subject; a mask image captured without the application of contrast agent is subtracted from an X-ray image of blood vessels at least partly filled with contrast agent, so as to obtain a subtraction angiography image. However, FIG. 1 shows a subtraction angiography image 100 obtained using relevant technology, and as shown in FIG. 1, a region 101 is an image of a partial region of the target region (e.g., the heart), in which an image of blood vessels can be observed; a region 102 is an image of a region surrounding the target region, the region 102 perhaps being a cavity or part of an organ of lower density (e.g. a lung containing air), so the image of the region 102 suffers from over-exposure. As can be seen, contrast between the region 102 and the region 101 is considerable, with a detrimental effect on normal imaging of the target region. One way of avoiding such over-exposure is for an operator to control one or more filters to move to the region 102 by manipulating a control lever manually, and manually control the orientation of the filter to enable it to block the region 102 to a large extent, reducing the exposure dose of the region 102 by filtering (blocking rays). However, this method is not only very demanding in terms of the operating skill of the operator, but also has low operating efficiency, because the operator might need to continuously change the position or orientation of the filter during the interventional surgery to adapt to surgical demands. Furthermore, this method requires pre-imaging of the test subject to acquire approximate information (e.g., position information) about the target region of the test subject; pre-imaging increases the ray dose received by the test subject, so it affects the test subject's health to a certain degree.

In view of the above, the present disclosure proposes a filter control method and apparatus for a medical imaging system, an imaging method for a medical imaging system, a medical imaging system, a non-transitory computer-readable storage medium, and a computer program product.

Aspects of the present disclosure are described in detail below with reference to the drawings.

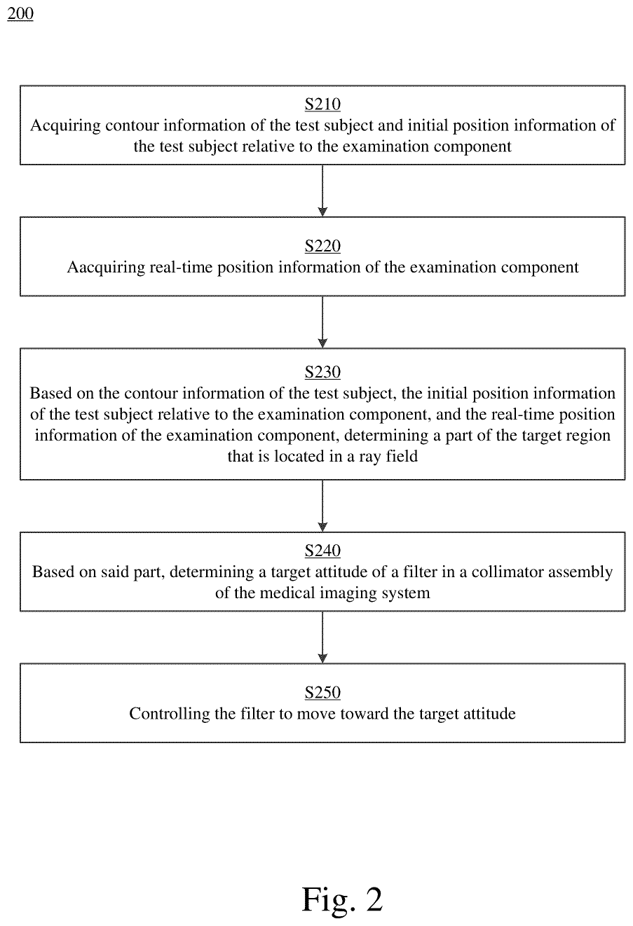

FIG. 2 shows a flow chart of a filter control method 200 for a medical imaging system according to an aspect of the present disclosure. The medical imaging system comprises at least one movable examination component for aligning a ray field with a target region of a test subject. The medical imaging system may be an X-ray imaging device (e.g., a C-arm X-ray imaging device), etc. The at least one movable examination component may comprise a radiation source (such as an X-ray tube), a detector for receiving rays emitted by the radiation source, or an examination table, etc. The test subject may be positioned on the examination table, and rays from the radiation source will pass through the test subject and the examination table to reach a detector and be received by the detector. The radiation source and examination table can move during actual use so as to be adjusted to suitable positions so that rays from the radiation source can pass through a target region to be tested on the test subject, and correspondingly, the detector will also move to a position corresponding to the radiation source, so as to receive the rays passing through the test subject.

As shown in FIG. 2, the filter control method 200 comprises:
step S210, acquiring contour information of the test subject and initial position information of the test subject relative to the examination component;
step S220, acquiring real-time position information of the examination component;
step S230, based on the contour information of the test subject, the initial position information of the test subject relative to the examination component, and the real-time position information of the examination component, determining a part of the target region that is located in a ray field;
step S240, based on said part, determining a target attitude of a filter in a collimator assembly of the medical imaging system; and
step S250, controlling the filter to move toward the target attitude.

In step S210, contour information of the test subject and initial position information of the test subject relative to the examination component are acquired. The test subject is, for example, a human body. The region to be tested of the test subject may be a region of the human body that needs to undergo radiographic examination, such as a hand, leg, chest, etc. For example, an image comprising the examination component and the test subject may be acquired by means of a sensor attached to the radiation source (e.g., a camera, such as a 3D camera or depth camera, or a laser scanner) or an additional sensor positioned independently of the radiation source (e.g., arranged on a wall of an examination room), in order to obtain from the image the contour information of the test subject and initial relative position information of the test subject relative to the examination component. Initial relative position information of the test subject relative to the examination component may also be measured by means of another sensor, e.g., a sensor arranged on the examination component. The initial relative position of the test subject relative to the examination component is initially (i.e., when the test subject has lain down at a suitable position on the examination table and no longer moves, i.e., at a fixed position) acquired relative position information of the test subject relative to the examination component at this moment. In some examples, the initial relative position information of the test subject relative to the examination component may be relative position information of the test subject relative to the radiation source and/or the detector (for example, relative position coordinates or another relative position relationship), or may be relative position information of the test subject relative to the examination table; these relative position relationships can all reflect the relative position relationship of the test subject relative to the various examination components (because the position of each examination component itself will also be acquired).

In an example, after obtaining the initial relative position information of the test subject relative to the examination component, relative positions of the test subject and the examination component in a virtual system model (described in detail below) for example can be updated on the basis of initial position information of the examination component (i.e. corresponding to the initial relative position information of the test subject relative to the examination component) and/or post-movement real-time position information thereof, so that the virtual system model can reflect the actual relative position situation of the test subject and the examination component, to facilitate subsequent determination of the target attitude of the filter.

In step S220, real-time position information of the examination component is acquired. The real-time position information of the examination component is position information of the examination component acquired in real time, wherein “acquired in real time” may refer to acquisition at intervals of a predetermined time, or acquisition at the same time as the position of the examination component changes, or acquisition when the examination component moves to a target position. Since the examination component is able to adjust its own position as required, the medical imaging system needs to acquire position information of the examination component in real time to ensure the accuracy of the position information of the examination component. The real-time position information of the examination component may be position information of the examination component acquired at the moment when step S220 is performed, or may be position information of the examination component acquired at the same moment that step S220 is performed. In summary, the real-time position information of the examination component can reflect the latest position information (which may have changed from the initial position of the examination component, or may remain the same as the initial position) of the examination component at the moment when step S220 is performed; in this way, it is possible to ensure that the actual relative position situation of the test subject and the examination component, i.e., the relative position situation fixed for performing exposure, is obtained.

In some examples, a direct 3D camera or a laser scanner may be used to capture an image of or scan the examination component to directly acquire real-time position information of the examination component. In other examples, real-time position information of the examination component may be acquired on the basis of initial position information and movement information of the examination component, wherein initial position information of each examination component in the medical imaging system, such as an initial relative position of each examination component, is known, and movement information of the examination component, such as a displacement and rotation angle of each examination component, can be obtained by means of a measuring unit such as a displacement sensor and angle sensor, or obtained by reading a movement instruction sent to the examination component.

In step S230, a part of the target region that is located in a ray field is determined on the basis of the contour information of the test subject, the initial position information of the test subject relative to the examination component, and the real-time position information of the examination component. For example, based on the initial position information of the test subject relative to the examination component and the real-time position information of the examination component, real-time position information of the test subject relative to the examination component (for example, relative position coordinates or another relative position relationship) can be determined. Since the initial position and orientation of the radiation source used for emitting rays in the examination component are known, the real-time position of the test subject relative to the ray field formed by the rays can also be determined. Further, with reference to the contour information of the test subject, it is possible to determine a part of the target region in the test subject that is located in the ray field (e.g., coordinates of the part of the target region in the test subject that is located in the ray field).

In step S240, based on the part of the target region in the test subject that is located in the ray field, a target attitude of a filter in a collimator assembly of the medical imaging system is determined. In an example, a target position of the filter may be determined, and at this target position, the filter may be positioned around the part of the target region that is located in the ray field and may at least partly block the passage of rays through an area surrounding the target region.

In step S250, according to the target attitude determined, the filter is controlled to move toward the target attitude.

In the aspect above, the target attitude of the filter in the collimator assembly of the medical imaging system can be determined automatically according to the contour information of the test subject, the initial position information of the test subject relative to the examination component, and the real-time position information of the examination component, etc. Thus, there is no need for an operator to manipulate a control lever manually to control the filter in the collimator assembly, so the target attitude of the filter that is finally determined is not restricted by the operator's skill or experience level, and has higher efficiency and accuracy. Furthermore, there is no need to perform pre-imaging of the test subject, so the radiation dose received by the test subject can be lowered, reducing harm to the test subject's health.

It will be understood that the “filter” herein may be sheet-like (e.g. a filter of any thickness), or may be another shape, as long as it is able to perform filtering (i.e. at least partly block rays).

According to some aspects, in the target attitude, the filter may be positioned at a periphery of the part of the target region that is located in the ray field. Thus, the filter is in just the right place to block rays passing through a region at the periphery of the target region without affecting the passage of rays through the target region.

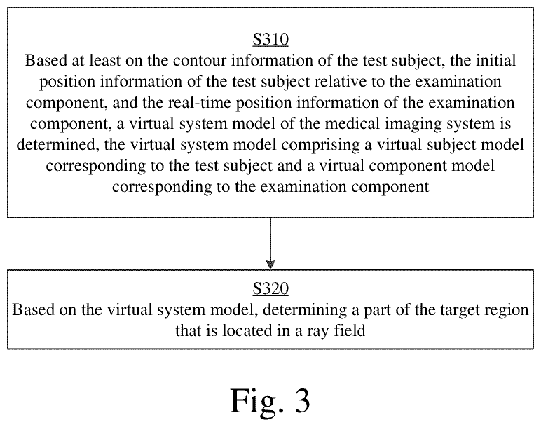

FIG. 3 shows a flow chart of a partial process of a filter control method for a medical imaging system according to an aspect of the present disclosure.

According to some aspects, as shown in FIG. 3, step S230 mentioned above may comprise:
step S310, based at least on the contour information of the test subject, the initial position information of the test subject relative to the examination component, and the real-time position information of the examination component, a virtual system model of the medical imaging system is determined, the virtual system model comprising a virtual subject model corresponding to the test subject and a virtual component model corresponding to the examination component; and
step S320, based on the virtual system model, determining a part of the target region that is located in a ray field.

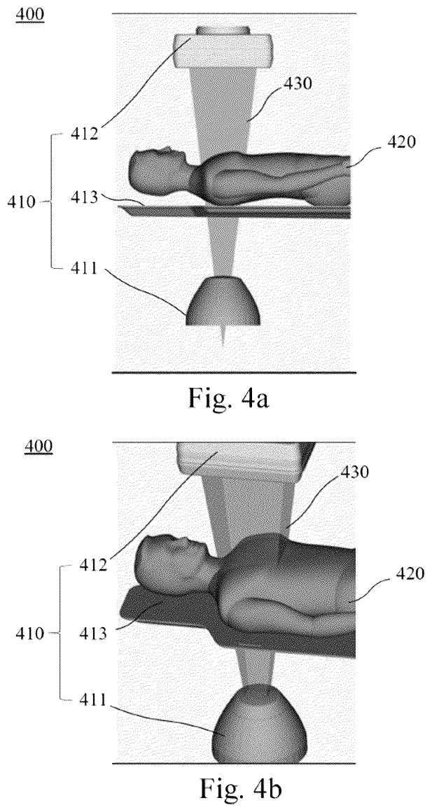

FIGS. 4a and 4b, respectively, show schematic drawings of virtual system models according to some exemplary aspects of the present disclosure, viewed from different angles. As shown in FIGS. 4a and 4b, the virtual system model 400 may comprise a virtual subject model 420 corresponding to the test subject, and a virtual component model 410 corresponding to the examination components. The virtual component model 410 corresponding to the examination components may comprise a virtual radiation source model 411 corresponding to the radiation source and a virtual examination table model 413 corresponding to the examination table, wherein movement of the radiation source and the examination table will affect which region of the test subject is in the ray field. Additionally, the virtual component model 410 corresponding to the examination components may further comprise a virtual detector model 412 corresponding to the detector; if the position of the radiation source changes, the position of the detector will change accordingly to receive the rays emitted by the radiation source. In some examples, position information and size information of the part of the target region of the test subject that is located in a ray field 430 may be determined on the basis of a relative positional relationship, reflected in the virtual system model 400, between the region to be tested of the test subject and the ray field 430. Alternatively, position information and size information of the part of the target region of the test subject that is located in the ray field 430 may be obtained by measurement directly from the virtual system model 400. As described above, position information and size information of the part of the target region of the test subject that is located in the ray field can be determined simply, efficiently and accurately by means of the virtual system model, thereby further improving the efficiency and accuracy of subsequent determination of the target attitude of the filter.

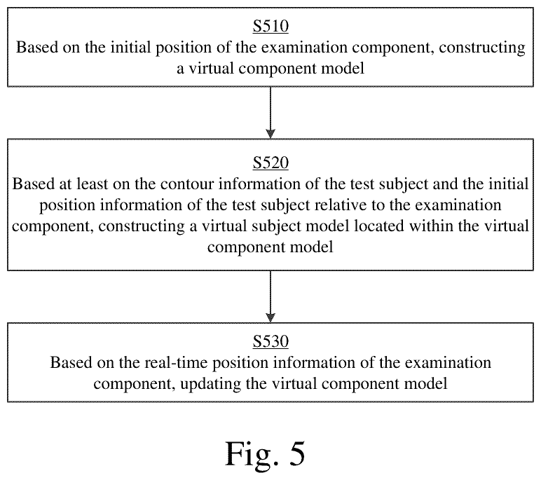

FIG. 5 shows a flow chart of a partial process of a filter control method for a medical imaging system according to an aspect of the present disclosure.

According to some aspects, as shown in FIG. 5, step S310 mentioned above may comprise:
step S510, based on the initial position of the examination component, constructing a virtual component model;
step S520, based at least on the contour information of the test subject and the initial position information of the test subject relative to the examination component, constructing a virtual subject model located within the virtual component model; and
step S530, based on the real-time position information of the examination component, updating the virtual component model.

In an example, the virtual component model may be constructed on the basis of the initial position of the examination component, or alternatively, the virtual component model may be a virtual model that is pre-stored in the medical imaging system; and the virtual subject model located within the virtual component model may be constructed at least on the basis of the contour information and initial relative position information of the test subject; and the virtual component model is updated on the basis of the real-time position information of the examination component. In other words, for example, in the case where a virtual component model relating to the examination component is pre-stored in the medical imaging system, the positions of various parts in the pre-stored virtual component model correspond to the initial positions of various components in the examination component; in this case, a virtual subject model corresponding to the contour of the test subject may be constructed on the basis of the contour information of the test subject, and based on the initial relative position of the test subject relative to the examination component, the virtual subject model may be placed in a corresponding position within the virtual component model. It should be understood here that in order to construct the virtual system model comprising the virtual subject model, the initial relative position information of the test subject relative to the examination component may be relative position information of the test subject relative to at least one of the detector, the radiation source and the examination table; all of these items of initial relative position information enable the virtual subject model to be placed in a position in the virtual component model that corresponds to the actual situation. Next, as shown in FIGS. 4a and 4b, after placing the virtual subject model 420 into the virtual component model 410, the virtual component model 410 can be updated on the basis of real-time position information of the examination component. In this way, it can be ensured that the relative positional relationship between the virtual subject model 420 and the virtual component model 410 in the virtual system model 400 remains consistent with the actual relative positional relationship between the test subject and the examination component, regardless of whether the examination component moves relative to its initial position.

According to some aspects, the virtual subject model may be obtained in the following way: acquiring a 3D contour of the test subject; acquiring an initial 3D virtual model of the target region; and merging the initial 3D virtual model with the 3D contour, to acquire a virtual anatomical model.

In an example, a 3D camera or a depth camera, or a laser scanner may be used to acquire the 3D contour of the test subject.

In an example, the initial 3D virtual model of the target region may be a 3D virtual model that is pre-stored in the medical imaging system; based on the choice of the operator, an initial 3D virtual model of a corresponding target region may be chosen from among pre-stored initial 3D virtual models of multiple target regions. As is known, in order to obtain optimal image quality in the process of subjecting a patient to image capture, an imaging protocol will generally be preset for a user in the medical imaging system, and the user can manually select this imaging protocol. Multiple imaging protocols are predefined for different imaging regions, patient dimensions, and patient postures. These imaging protocols are generally related to operating parameters to be realized by a control apparatus of the medical imaging system, such as X-ray generator frame rate, radiation dose, noise processing, and signal post-processing, etc., enabling the user to provide an image capture mode giving the clearest images on the basis of the target region of the patient that currently needs to undergo image capture. Such an imaging protocol, especially in medical imaging systems, is also called an organ program (OGP). In some examples, based on an OGP selected by the user, an initial 3D virtual model of a target region corresponding to the OGP may be determined.

In an example, based on the body type or weight of the test subject, an initial 3D virtual model of a target region of corresponding size may be acquired, and the initial 3D virtual model of the target region may be merged to a corresponding position of a 3D contour according to the body type or weight of the test subject, so as to acquire a virtual anatomical model.

According to some aspects, the examination components may comprise a collimator assembly and an examination table for carrying the test subject, wherein the collimator assembly may comprise a filter for limiting the radiation scope of rays (e.g., a rectangular filter, wedge-shaped filter, or finger-shaped filter), and the acquisition of real-time position information of the examination component may comprise:
acquiring initial position information of the collimator assembly and the examination table;
acquiring real-time position information of the collimator assembly and the examination table; and
determining real-time position information of the collimator assembly and the examination table on the basis of the initial position information and real-time position information of the collimator assembly and the examination table.

In an example, the initial position information of the collimator assembly and the examination table may be known, and the real-time position information of the collimator assembly and the examination table may be position information of the examination components acquired at the moment when step S220 is performed, or may be position information of the examination components acquired at the same moment that step S220 is performed. In summary, the real-time position information of the collimator assembly and the examination table can reflect the latest position information of the collimator assembly and the examination table at the moment when step S220 is performed; in this way, it is possible to ensure that the actual relative position situation of the test subject and the collimator assembly and examination table, i.e., the relative position situation fixed for performing exposure, is obtained.

According to some aspects, the filter may comprise multiple types of filters, and the filter control method for a medical imaging system may further comprise:
acquiring an inputted operation type for the target region; and
based on the inputted operation type, determining at least one corresponding filter from the multiple types of filter.

As stated above, multiple organ programs (OGP) are predefined for different imaging regions, patient dimensions, and patient postures. The operation type may be defined in an organ program; during actual use, the operator may, based on an organ/region to be tested, etc., select a corresponding organ program (OGP) via a human-machine interactive device (such as a touch screen, a keyboard or a mouse, etc.), thereby inputting the operation type for the target region. Correspondingly, based on the selected organ program, the medical imaging system may determine at least one corresponding filter from the multiple types of filters automatically.

According to some aspects, the operation type may comprise organ vascular imaging, and the multiple types of filter may comprise a wedge-shaped filter and a finger-shaped filter. In an example, organ vascular imaging may comprise at least one of cardiovascular imaging, cerebrovascular imaging and vascular imaging of the four limbs.

In an example, when the operation type is interventional surgery on the heart, the wedge-shaped filter may be determined for filtering a periphery of the heart. In an example, when the operation type is an imaging process for the blood vessels of the lower limbs, the finger-shaped filter may be determined for filtering a gap between the two lower limbs.

According to some aspects, the target attitude may comprise a target position and a target orientation, and step S250 may comprise at least one of the following: controlling the filter to move toward the target position; and controlling the filter to rotate toward the target orientation.

In an example, it is possible to control the filter to move toward the target position or control the filter to rotate toward the target orientation. In an example, it is possible to both control the filter to move toward the target position, and also control the filter to rotate toward the target orientation. By controlling the movement and rotation of the filter, the position or orientation of the filter can be finely adjusted so as to control the filter to reach the desired attitude more accurately.

According to some aspects, the filter control method for a medical imaging system may further comprise:
in response to having controlled the filter to move toward a target attitude, acquiring an actual attitude of the filter; and
in response to determining that a difference between the actual attitude and target attitude of the filter exceeds a preset permitted range, issuing error prompt information.

For example, the controller may send a target attitude instruction to a filter execution mechanism (e.g., an electric motor) so as to control the filter to move toward the target attitude. When movement has ended, the controller may acquire a current position and rotation angle of the electric motor from the filter execution mechanism to serve as the actual attitude of the filter. By issuing error prompt information upon determining that the difference between the actual attitude and target attitude of the filter exceeds the preset permitted range, the operator can be promptly informed that the filter is not in the desired attitude so as to take remedial measures.

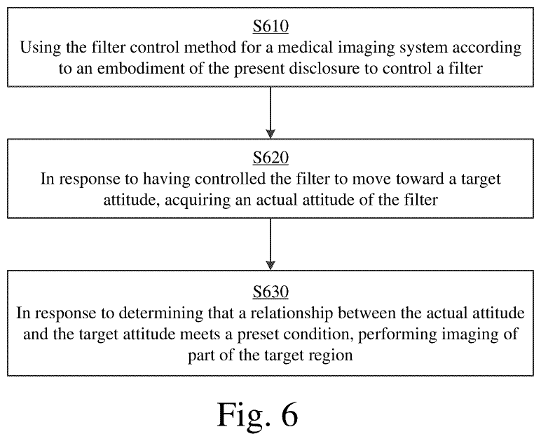

According to another aspect of the present disclosure, an imaging method for a medical imaging system is provided. FIG. 6 shows a flow chart of an imaging method 600 for a medical imaging system according to an aspect of the present disclosure.

As shown in FIG. 6, the imaging method 600 comprises:
step S610, using the filter control method for a medical imaging system according to an aspect of the present disclosure to control a filter;
step S620, in response to having controlled the filter to move toward a target attitude, acquiring an actual attitude of the filter; and
step S630, in response to determining that a relationship between the actual attitude and the target attitude meets a preset condition, performing imaging of part of the target region.

In an example, the controller may send a target attitude instruction to a filter execution mechanism (e.g. an electric motor), so as to control the filter to move toward the target attitude. When movement has ended, the controller may acquire a current position and rotation angle of the electric motor from the filter execution mechanism to serve as the actual attitude of the filter. Upon determining that the relationship between the actual attitude and the target attitude meets the preset condition, imaging of part of the target region is performed. In an example, the relationship between the actual attitude and the target attitude meeting the preset condition may comprise a difference value between coordinates of an actual position and coordinates of a target position of the filter being less than a preset threshold, and/or a difference value between an actual orientation and a target orientation of the filter being less than a preset threshold.

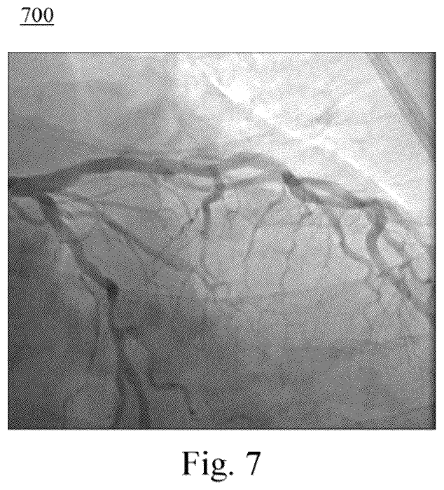

FIG. 7 shows a subtraction angiography image 700 obtained using the imaging method according to an aspect of the present disclosure. Taking subtraction angiography as an example, using the imaging method 600, when it is determined that the relationship between the actual attitude and the target attitude meets the preset condition, the subtraction angiography image 700 may be obtained by performing imaging of part of the target region. As can be seen, compared with subtraction angiography image 100, subtraction angiography image 700 is of higher quality; a region surrounding the target region (e.g., the heart) does not suffer from over-exposure, and the distribution of blood vessels in the target region can be clearly observed in the entire subtraction angiography image 700. Thus, by means of the imaging method 600, there is no need for an operator to manipulate a control lever manually to control the filter in the collimator assembly, high-quality imaging can be achieved with higher efficiency, and the radiation dose received by the test subject can be reduced.

According to some aspects, the medical imaging system may further comprise a ray emitter as the radiation source, and step S630 may comprise:
in response to determining that a relationship between the actual attitude and the target attitude meets a preset condition, determining a conducting state of a ray emission switch of the ray emitter; and
in response to determining that the ray emission switch is ON, performing imaging of part of the target region.

The ray emission switch may, for example, be controlled by a foot pedal; when the operator steps on the foot pedal, the ray emission switch of the ray emitter switches ON; when the operator releases the foot pedal, the ray emission switch of the ray emitter switches OFF.

According to some aspects, the imaging method 600 for a medical imaging system may further comprise:
in response to determining that the ray emission switch is OFF, re-determining a target attitude of the filter in the collimator assembly of the medical imaging system.

The position or orientation of one or more components in the medical imaging system might change continuously during operation, so when the ray emission switch is OFF, the operator might have already released the foot pedal and is currently controlling one or more components in the medical imaging system to move (e.g. controlling the C-arm to rotate); by re-determining a target attitude of the filter in the collimator assembly of the medical imaging system, it is possible to avoid exposing the test subject to radiation at a time when this is not desired.

According to another aspect of the present disclosure, a filter control apparatus for a medical imaging system is provided. The medical imaging system comprises at least one movable examination component for aligning a ray field with a target region of a test subject.

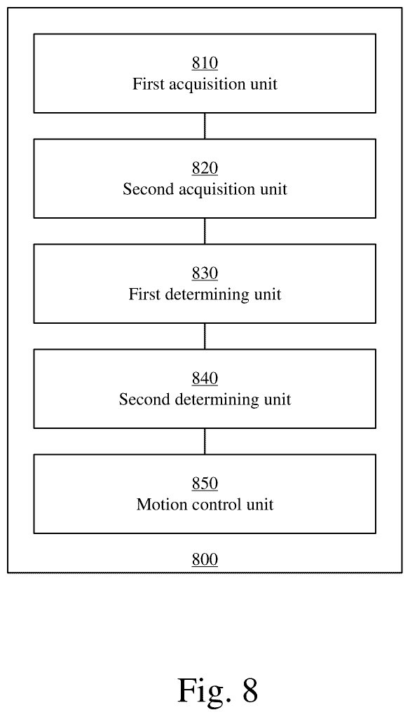

FIG. 8 shows a schematic block diagram of a filter control apparatus 800 for a medical imaging system according to an aspect of the present disclosure. As shown in FIG. 8, the filter control apparatus 800 comprises:
a first acquisition unit 810, configured to acquire contour information of the test subject and initial position information of the test subject relative to the examination component;
a second acquisition unit 820, configured to acquire real-time position information of the examination component;
a first determining unit 830, configured to determine a part of the target region that is located in a ray field on the basis of the contour information of the test subject, the initial position information of the test subject relative to the examination component, and the real-time position information of the examination component;
a second determining unit 840, configured to determine a target attitude of a filter in a collimator assembly of the medical imaging system on the basis of said part; and
a motion control unit 850, configured to control the filter to move toward the target attitude.

It should be understood that the units of the filter control apparatus 800 shown in FIG. 8 may correspond to the steps in the filter control method 200 described with reference to FIG. 2. Thus, the operations, features, and advantages described above in relation to the filter control method 200 likewise apply to the filter control apparatus 800 and the units comprised therein. For conciseness, some operations, features and advantages are not described again here.

According to another aspect of the present disclosure, an imaging apparatus (not shown) for a medical imaging system is provided, comprising:
a filter control unit configured to use the filter control apparatus 800 to control a filter;
an actual attitude acquisition unit, configured to acquire an actual attitude of the filter in response to having controlled the filter to move toward a target attitude; and
an imaging unit configured to perform imaging of part of the target region in response to determining that a relationship between the actual attitude and the target attitude meets a preset condition.

According to another aspect of the present disclosure, a medical imaging system is provided, comprising: at least one processor, and a memory in communicative connection with the at least one processor, wherein the memory stores a computer program which, when executed by the at least one processor, realizes the method according to an aspect of the present disclosure.

According to another aspect of the present disclosure, a non-transitory computer-readable storage medium storing a computer program is provided, wherein the computer program, when executed by a processor, realizes the method according to an aspect of the present disclosure.

According to another aspect of the present disclosure, a computer program product is provided, comprising a computer program, wherein the computer program, when executed by a processor, realizes the method according to an aspect of the present disclosure.

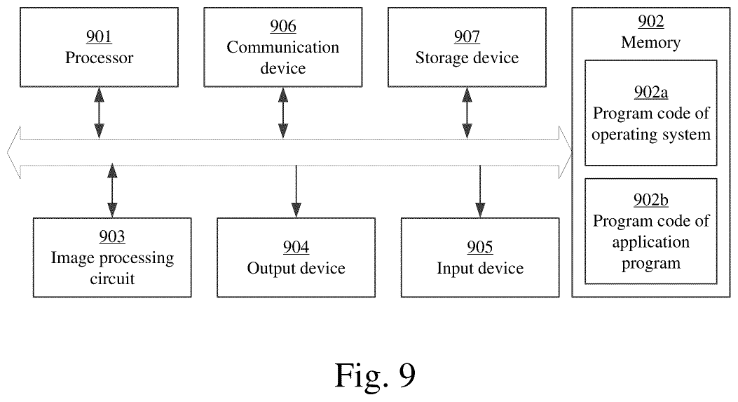

FIG. 9 is a block diagram showing an example of an electronic device 900 according to an exemplary aspect of the present disclosure. It must be explained that the structure shown in FIG. 9 is merely an example; depending on the specific manner of implementation, the electronic device of the present disclosure may only comprise one or more of the constituent parts shown in FIG. 9.

The electronic device 900 may, for example, be a general-purpose computer (e.g., a laptop computer, a tablet computer, or various other types of computer), a mobile phone, or a personal digital assistant. According to some aspects, the electronic device 900 may be a cloud computing device and a smart device. According to some aspects, the electronic device 900 may be the medical imaging device described above, e.g., the C-arm X-ray imaging device.

According to some aspects, the electronic device 900 may be configured to subject an image, etc., to processing and transmit a result of the processing to an output device so that it is provided to a user. The output device may, for example, be a display screen, a device comprising a display screen, or another output device. For example, the electronic device 900 may be configured to subject the image to target detection and transmit a result of target detection to a display device for display; the electronic device 900 may also be configured to subject the image to enhancement processing and transmit a result of enhancement to a display device for display.

The electronic device 900 may comprise an image processing circuit 903; the image processing circuit 903 may be configured to subject the image to various types of image processing. The image processing circuit 903 may, for example, be configured to subject the image to at least one of the following types of image processing: noise reduction, geometric correction, feature extraction, detection and/or identification of objects in the image, and enhancement processing. The image processing circuit 903 may use custom hardware and/or may be realized using hardware, software, firmware, middleware, microcode, hardware description language, or any combination thereof. For example, one or more of the various circuits mentioned above may be realized by using assembly language or hardware programming language (such as VERILOG, VHDL, C++) to program hardware (e.g., a programmable logic circuit comprising a field programmable gate array (FPGA) and/or a programmable logic array (PLA)), using logic and algorithms according to the present disclosure.

According to some aspects, the electronic device 900 may further comprise an output device 904; the output device 904 may be any type of device used to present information and may include, but is not limited to, a display screen, a terminal with display functionality, earphones, a loudspeaker, a vibration device and/or a printer, etc.

According to some aspects, the electronic device 900 may further comprise an input device 905; the input device 905 may be any type of device for inputting information to the electronic device 900 and may include, but is not limited to, various types of sensor, mouse, keyboard, touch screen, push button, control lever, microphone and/or remote controller, etc.

According to some aspects, the electronic device 900 may further comprise a communication device 906; the communication device 906 may be any type of device or system enabling communication with an external device and/or with a network and may include, but is not limited to, a modem, a network card, an infrared communication device, a wireless communication device and/or a chipset, e.g. a Bluetooth device, a 802.11 device, a WiFi device, a WiMax device, a cellular communication device and/or similar.

According to some aspects, the electronic device 900 may further comprise a processor 901. The processor 901 may be any type of processor and may include, but is not limited to, one or more general-purpose processors and/or one or more dedicated processors (e.g., special processing chips). The processor 901 may, for example, be, but is not limited to being, a central processing unit (CPU), a graphics processing unit (CPU), or various types of dedicated artificial intelligence (AI) computing chips, etc. In an example in which the electronic device 900 may be a magnetic resonance scanning imaging device, the processor 901 may be a processor of a master control computer of the magnetic resonance scanning imaging device.

The electronic device 900 may further comprise a working memory 902 and a storage device 907. The processor 901 may be configured to be able to acquire and execute computer-readable instructions stored in the working memory 902, the storage device 907 or another computer-readable medium, such as program code of an operating system 902a, program code of an application program 902b, etc. The working memory 902 and storage device 907 are examples of computer-readable storage media used to store instructions, and the stored instructions may be executed by the processor 901 to implement the various functions described above. The working memory 902 may comprise both a volatile memory and a non-volatile memory (e.g., RAM, ROM, etc.). The storage device 907 may comprise a hard disk drive, solid state drive, removable media, including external and removable drives, memory cards, flash memory, floppy disks, optical disks (e.g., CD, DVD), storage arrays, network attached storage, storage area networks, etc. The working memory 902 and storage device 907 may both be collectively referred to as memory or computer-readable storage media herein and may be non-transitory media capable of storing computer-readable, processor-executable program instructions as computer program code, which may be executed by the processor 901 as a specific device configured to implement the operations and functions described in the examples herein.

According to some aspects, the processor 901 may perform control and scheduling of at least one of the image processing circuit 903 and various other apparatuses and circuits comprised in the electronic device 900. According to some aspects, at least some of the constituent parts in FIG. 9 may be connected to and/or communicate with each other via a bus 908.

Software elements (programs) may be located in the working memory 902, including but not limited to an operating system 902a, one or more application program 902b, a driver, and/or other data and code.

According to some aspects, instructions for performing the abovementioned control and scheduling may be comprised in the operating system 902a or one or more application programs 902b.

According to some aspects, instructions for executing the method steps of the present disclosure may be included in the one or more application program 902b, and the modules of the electronic device 900 mentioned above may be realized by the processor 901 reading and executing the instructions of the one or more application program 902b. In other words, the electronic device 900 may comprise the processor 901 and a memory storing a program (e.g., the working memory 902 and/or storage device 907), the program comprising instructions which, when executed by the processor 901, cause the processor 901 to perform the method according to various aspects of the present disclosure.

According to some aspects, some or all of the operations performed by the image processing circuit 903 may be realized by the processor 901 reading and executing the instructions of one or more application programs 902b.

Executable code or source code of instructions of software elements (programs) may be stored in a non-transitory computer-readable storage medium (e.g., the storage device 907) and, when executed, may be stored in the working memory 902 (possibly compiled and/or installed). Thus, the present disclosure provides a computer-readable storage medium storing a program, the program comprising instructions which, when executed by a processor of an electronic device, cause the electronic device to perform the method according to various aspects of the present disclosure. According to another aspect, executable code or source code of instructions of software elements (programs) may also be downloaded from a remote location.

It should also be understood that various changes in form may be carried out according to particular requirements. For example, it is also possible to use custom hardware, and/or it is possible to use hardware, software, firmware, middleware, microcode, hardware description language, or any combination thereof to realize each circuit, unit, module, or element. For example, some or all of the circuits, units, modules, or elements contained in the disclosed methods and devices may be realized by using assembly language or hardware programming language (such as VERILOG, VHDL, C++) to program hardware (e.g., a programmable logic circuit comprising a field programmable gate array (FPGA) and/or a programmable logic array (PLA)), using logic and algorithms according to the present disclosure.

According to some aspects, the processor 901 in the electronic device 900 may be distributed on a network. For example, one processor may be used to perform some processing, and at the same time, other processing may be performed by another processor remote from said one processor. Other modules of the electronic device 900 may also be similarly distributed. Thus, the electronic device 900 may be interpreted as being a distributed computing system that performs processing at multiple locations. The processor 901 of the electronic device 900 may also be a processor of a cloud computing system or a processor integrated with a blockchain.

Although aspects or examples of the present disclosure have already been described with reference to the drawings, it should be understood that the abovementioned methods, systems, and devices are merely exemplary aspects or examples, and the scope of the present invention is not limited by these aspects or examples, instead being defined solely by the granted claims and the equivalent scope thereof. Various key elements in the aspects or examples may be omitted or may be replaced by equivalent key elements thereof. In addition, the steps may be executed in an order different from that described in the present disclosure. Furthermore, various key elements in the aspects or examples may be combined in various ways. Importantly, as technology evolves, many key elements described here may be replaced by equivalent key elements appearing after the present disclosure.

1. A filter control method for a medical imaging system that includes at least one examination component operable to align a ray field with a target region of a test subject, the method comprising:
acquiring contour information of the test subject and initial position information of the test subject relative to the examination component;
acquiring real-time position information of the examination component;
determining a part of the target region that is located in the ray field based on the contour information of the test subject, the initial position information of the test subject relative to the examination component, and the real-time position information of the examination component;
determining a target attitude of a filter in a collimator assembly of the medical imaging system based on said part; and
controlling the filter to move toward the target attitude.

2. The method as claimed in claim 1, wherein the step of determining a part of the target region that is located in the ray field based on the contour information of the test subject, the initial position information of the test subject relative to the examination component, and the real-time position information of the examination component, comprises:
determining a virtual system model of the medical imaging system at least based on the contour information of the test subject, the initial position information of the test subject relative to the examination component, and the real-time position information of the examination component, the virtual system model comprising a virtual subject model corresponding to the test subject and a virtual component model corresponding to the examination component; and
determining a part of the acquired target region that is located in the ray field based on the virtual system model.

3. The method as claimed in claim 2, wherein the step of determining a virtual system model of the medical imaging system at least based on the contour information of the test subject, the initial position information of the test subject relative to the examination component, and the real-time position information of the examination component, comprises:
constructing the virtual component model based on an initial position of the examination component;
constructing the virtual subject model located within the virtual component model at least based on the contour information of the test subject and the initial position information of the test subject relative to the examination component; and
updating the virtual component model based on the real-time position information of the examination component.

4. The method as claimed in claim 2, wherein the virtual subject model is obtained by:
acquiring a three-dimensional (3D) contour of the test subject;
acquiring an initial 3D virtual model of the target region; and
merging the initial 3D virtual model with the 3D contour, to acquire a virtual anatomical model.

5. The method as claimed in claim 1, wherein the examination component comprises the collimator assembly and an examination table for carrying the test subject, and wherein the step of acquiring real-time position information of the examination component comprises:
acquiring initial position information of the collimator assembly and the examination table;
acquiring real-time position information of the collimator assembly and the examination table; and
determining real-time position information of the collimator assembly and the examination table based on the initial position information and real-time position information of the collimator assembly and the examination table.

6. The method as claimed in claim 1, wherein the target attitude comprises a target position and a target orientation, and wherein the step of controlling the filter to move toward the target attitude comprises:
controlling the filter to move toward the target position; or
controlling the filter to rotate toward the target orientation.

7. The method as claimed in claim 1, wherein, in the target attitude, the filter is positioned at a periphery of the part of the target region that is located in the ray field.

8. The method as claimed in claim 1, further comprising:
in response to having controlled the filter to move toward the target attitude, acquiring an actual attitude of the filter; and
in response to determining that a difference between the actual attitude and the target attitude of the filter exceeds a preset permitted range, issuing error prompt information.

9. An imaging method for a medical imaging system, comprising:
using the method as claimed in claim 1 to control the filter;
in response to having controlled the filter to move toward the target attitude, acquiring an actual attitude of the filter; and
in response to determining that a relationship between the actual attitude and the target attitude meets a preset condition, performing imaging of said part of the target region.

10. The method as claimed in claim 9, wherein the medical imaging system further comprises a ray emitter, and wherein the step of performing imaging of said part of the target region in response to determining that a relationship between the actual attitude and the target attitude meets a preset condition, comprises:
in response to determining that a relationship between the actual attitude and the target attitude meets a preset condition, determining a conducting state of a ray emission switch of the ray emitter; and
in response to determining that the ray emission switch is ON, performing imaging of said part of the target region.

11. The method as claimed in claim 10, further comprising:
in response to determining that the ray emission switch is OFF, re-determining a target attitude of the filter in the collimator assembly of the medical imaging system.

12. A filter control apparatus for a medical imaging system that includes at least one examination component operable to align a ray field with a target region of a test subject, the filter control apparatus comprising:
a first acquisition unit configured to acquire contour information of the test subject and initial position information of the test subject relative to the examination component;
a second acquisition unit configured to acquire real-time position information of the examination component;
a first determining unit configured to determine a part of the target region that is located in the ray field based on the contour information of the test subject, the initial position information of the test subject relative to the examination component, and the real-time position information of the examination component;
a second determining unit configured to determine a target attitude of a filter in a collimator assembly of the medical imaging system based on the part; and
a motion control unit configured to control the filter to move toward the target attitude.

13. A medical imaging system, comprising:
at least one processor; and
a memory in communicative connection with the at least one processor, wherein the memory stores a computer program which, when executed by the at least one processor, realizes the method as claimed in claim 1.

14. A non-transitory computer-readable storage medium storing a computer program, wherein the computer program, when executed by a processor, realizes the method as claimed in claim 1.
